(19) United States
(12) Patent Application Publication
(10) Pub. No.: US 2012/0276596 A1
(43) Pub. Date: Nov. 1, 2012

(54) MODIFIED BIOMATERIAL, USES THEREOF AND MODIFICATION METHODS

(75) Inventors: Jaakko Pere, Vtt (FI); Maria Smolander, Vtt (FI); Harry Boer, Vtt (FI)

(73) Assignee: TEKNOLOGIAN TUTKIMUSKESKUS VTT, Espoo (FI)

(21) Appl. No.: 13/515,942

(22) PCT Filed: Dec. 15, 2010
(86) PCT No.: PCT/FI2010/051037

§ 371 Date: Jun. 14, 2012

(30) Foreign Application Priority Data
Dec. 15, 2009 (FI) 20096326

(51) Int. Cl. C08B 37/06; C09D 105/06; C12P 19/04

(52) U.S. Cl. 435/101; 536/2; 106/217.7

(57) ABSTRACT

The present invention relates to the fields of biomass technology, and more precisely to applications of packaging, and coating products for food and cosmetics. The present invention relates to a method of modifying a polymeric polysaccharide matrix and to a method of coating a product to impart new properties to the product. The present invention further relates to a modified polymeric polysaccharide matrix, to a product being coated with a modified polymeric polysaccharide matrix and uses thereof.

### FIELD OF THE INVENTION

The present invention relates to the fields of biomass technology, and more precisely to applications of packaging, and coating products for food and cosmetics. The present invention relates to a method of modifying a polymeric polysaccharide matrix and to a method of coating a product to impart new properties to the product. The present invention further relates to a modified polymeric polysaccharide matrix, to a product being coated with a modified polymeric polysaccharide matrix and uses thereof.

### BACKGROUND OF THE INVENTION

Due to increased consumption and expansive assortment of products, a need for specific packaging materials has increased during the decades. A non-stop development of the items to be packed and continuously varying requirements of the packaging materials challenge the packaging industry.

Many foods require specific conditions to sustain their freshness and overall quality during storage. Hence, our foods are being packed by using methods and materials, which ensure optimum quality, safety and facility of the food product in question. To ensure e.g. freshness, physical quality and microbial safety of the food product during storage, the packaging material needs to have certain barrier properties.

The conventional approaches to produce high barrier films for packaging of food are to use multilayers of different films or synthetic, plastic or metal coatings on packaging materials. However, there is a growing need for pro-environmental solutions in packaging industry in order to reduce the environmental load. Furthermore, reduction of production costs may be sought for example by recycling materials, such as by-products of food industry.

An alternative for synthetic, plastic or metal packaging material is natural polymers. Examples of natural polymers are polysaccharides, such as pectin, hemicelluloses, cellulose and starch, and proteins, such as casein, gluten from wheat and corn, whey, collagen, keratin and soy.

From the group of polysaccharides, hemicelluloses and pectins have received attention in films and coatings area because they provide a potential to control transfer of for example oxygen, aroma, oil, and flavour compounds.

Pectins belong to a group of hemicelluloses, i.e. non-cellulosic, non starch plant polysaccharides. Pectin is an acidic, structural heteropolysaccharide contained in the primary cell walls of terrestrial plants. It is also present in the middle lamella between plant cells where it helps to bind cells together. For industrial purposes pectin is mainly extracted from apple pomace, citrus fruits and sugar beet chips and it is used in food or pharmaceuticals as a gelling agent, stabilizer or a source of dietary fiber.

Pectin has a complex structure. Pectin, when extracted from higher plants, contains smooth (linear) regions and hairy, branched regions. The linear, smooth regions are made up of α-(1-4)-linked D-galacturonic acid residues, some of which are methylesterified at C-6 position and may be acetylated at C-2 or C-3 positions. The hairy region contains a backbone of the repeating disaccharide (→4)-α-D-GalpA-(→2)-α-L-Rhap-(→). The Rhap residues are substituted at C-4 with neutral and acidic oligosacchadide side chains composed of mainly arabinose and galactose and depending on pectin source also fucose and glucuronic acid. These arabinose and galactose residues in the neutral sugar side chains are in some cases (e.g. in sugar beet pectin) substituted by ferulic acid residues linked at C-2 (arabinose) or C-6 (galactose) positions. In the plant cell wall pectin contains also a substituted galacturonan (rhamnogalacturonan II,RG-II). The backbone of RG-II is composed of at least seven 1,4-linked α-D-GalpA residues, to which structurally different oligosaccharide side chains are attached. RG-II is greatly reduced or absent in commercial pectins due to the extraction and purification procedures used.

The degree of esterification determines the solubility of pectin and its gelling and film forming properties and hence its industrial applicability to a large extent. The degree of methylesterification varies with the origin of the plant source and the processing conditions e.g. storage, extraction, isolation and purification. Commercial pectins are graded to low (D. E. <50%) and high (D. E. >50%) methoxyl pectins. For special needs pectins can be further modifled by enzymatic means, e.g. molar mass can be reduced by polygalacturonases and D. E. can be tuned by pectin methylesterase.

The chemical formula of pectins is shown below.

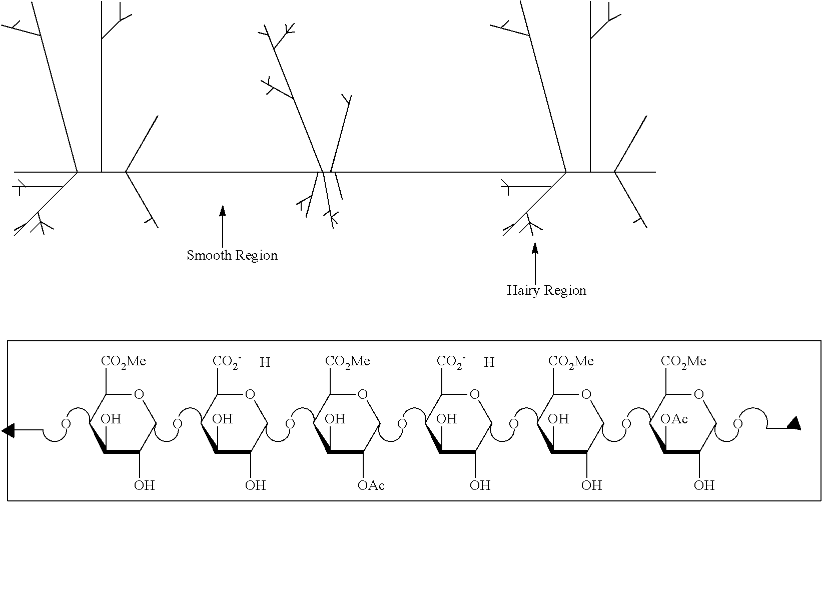

Xylan is the most important component of hemicellulose. Xylans are major components in the primary cell wall of monocots and are found in smaller amounts in the primary wall of dicots. Xylans have a backbone of β-1,4-linked xylose residues. In arabinoxylan the backbone is substituted by arabinofuranosyl residues attached to O-2 or O-3 of xylosyl residues. The xylan backbone is substituted by α-linked 4-O-methyl-β-D-glucopyranosyl uronic acid on O-2 of xylosyl residues and acetyl esters on O-2 or O-3. The degree of chain substitution determines the degree of solubility of the xylan in question. Primary cell walls of gramineous monocots contain arabinoxylan esterified by ferulic and p-coumaric acids. Feruloylation and p-coumaraylation occur at O-5 of the arabinofuranosyl side chain of xylan.

Due to the hydrophilic nature of polysaccharides, their gas barrier properties are very much dependent on the humidity conditions. The gas permeability of polysaccharide materials may increase manifold when humidity increases (Natanya Hansen & David Plackett. 2008. Sustainable Films and Coatings from Hemicelluloses: A Review. Biomacromolecules 9: 1493-1505). In the presence of moisture, the macromolecule chains become more mobile which leads to a substantial increase in oxygen permeability. In general, non-ionic polysaccharide films appear to have higher oxygen permeabilities than protein films. This may be related to their less polar nature and less linear structure, leading to lower cohesive energy density and higher free volume (Khwaldia, K., Perez, C., Banon, S., Desobry, S. & Hardy, J. Milk proteins for edible films and coatings. Critical Reviews in Food Science and Nutrition, Vol. 44 (2004) 4, p. 239251).

The major drawbacks in barrier properties of polysaccharide coatings have been overcome by blending or laminating the polysaccharides with other bio based materials, such as polyhydroxyalkanoate (PHA) and polylactic acid (PLA). Another way to modify polysaccharide properties is by chemical modification.

Grease resistance is an important characteristic of packaging materials used with products containing fat or oil. Generally, polysaccharide films are expected to be highly grease resistant due to their substantial hydrophilicity (Innovations in Food Packaging. Jung H. Han (ed) Food Science and Technology, International Series, Elsevier Ltd, London, 2005). However, grease resistance properties of polysaccharides can also be modified for example by chemical modification.

Current approaches to extend functional and mechanical properties of natural polymer films, include (i) incorporation of hydrophobic compounds, such as lipids in the film forming solutions; (ii) optimization of the interactions between polymers (protein-protein interactions, charge-charge electrostatic complexes between proteins and polysaccharides) and (iii) formation of crosslinks through physical, chemical, or enzymatic treatments or irradiation (Ouattara B. et al. 2002, Radiation Physics and Chemistry, Vol. 63 (3-6), 821-825).

For example, polysaccharides have been combined with proteins to form composite films. Examples include films from methylcellulose and zein, propylene glycol alginate and soy protein isolate, hydroxypropyl methylcellulose with protein isolate of Pistacia terebinthus, alginate or pectin with whey protein or caseinate, starch and zein, and starch and sodium caseinate (Yada R. Y., Proteins in Food Processing. Woodhead Publishing, http://www.knovel.com/knovel2/Toc.jsp?BookID=1221&Vertical ID=).

Furthermore, publication WO 98/22513 A1 describes production of gels by pectin cross-linking, and publication WO 9603546 A1 describes a process for the manufacture of a lignocellulose-based product by treating the lignocellulosic material and a phenolic polysaccharide with an enzyme capable of catalyzing the oxidation of phenolic groups. JP 05117591 A describes compositions having features similar to natural Japanese lacquer and comprising a vegetable mucous substance, such as pectin and oxidizing enzymes.

However, the present invention provides novel methods for modifying the polymeric polysaccharide matrixes and furthermore, for improving the barrier properties and/or mechanical properties of the polymeric polysaccharide matrixes. The polymeric polysaccharide matrixes of the present invention are useful for example in food and cosmetics packaging.

### BRIEF DESCRIPTION OF THE INVENTION

The present invention resides in the surprising finding that the properties of a polymeric polysaccharide matrix can be advantageously modified by combining cross-linking with functionalization, i.e. the addition of functional groups to the cross-linked polymeric polysaccharide or cross-linking the functionalized polymeric polysaccharides. The functional groups may e.g. be hydrophobic groups, whereby excellent barrier properties are obtained.

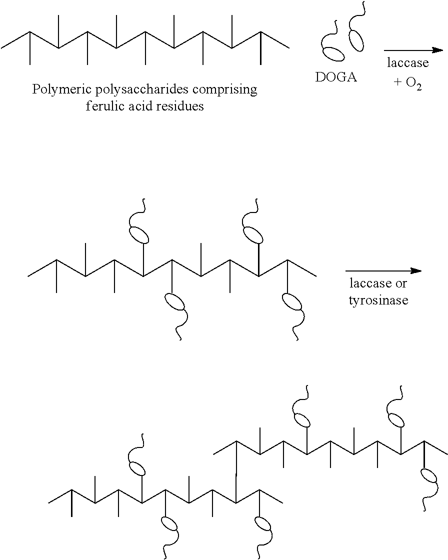

The present invention relates to a method of modifying a polymeric polysaccharide matrix, said method comprising
cross-linking polymeric polysaccharides in the matrix, and
functionalizing the polymeric polysaccharides by oxidizing ferulic acids of the polymeric polysaccharides, and contacting the oxidized polymeric polysaccharides with a hydrophobic modifying agent containing at least one first site, which is reactive with the oxidized ferulic acids, and at least one second site, which provides desired properties to the polymeric polysaccharide matrix,
whereby a modified polymeric polysaccharide matrix is obtained.

The present invention also relates to a method of coating a product, said method comprising
providing a polymeric polysaccharide matrix,
cross-linking polymeric polysaccharides in the matrix,
functionalizing the polymeric polysaccharides by oxidizing ferulic acids of the polymeric polysaccharides, and contacting the oxidized polymeric polysaccharides with a hydrophobic modifying agent containing at least one first site, which is reactive with the oxidized ferulic acids, and at least one second site, which provides desired properties to the polymeric polysaccharide matrix to obtain a modified polymeric polysaccharide matrix, and
coating the product with the modified polymeric polysaccharide matrix.

Furthermore, the present invention relates to a method of improving barrier or mechanical properties of a polymeric polysaccharide matrix or product, said method comprising
cross-linking polymeric polysaccharides in the matrix, and
functionalizing the polymeric polysaccharides by oxidizing ferulic acids of the polymeric polysaccharides, and contacting the oxidized polymeric polysaccharides with a hydrophobic modifying agent containing at least one first site, which is reactive with the oxidized ferulic acids, and at least one second site, which provides desired properties to the polymeric polysaccharide matrix, and
optionally coating the product with the modified polymeric polysaccharide matrix.

Furthermore, the present invention relates to a modified polymeric polysaccharide matrix comprising cross-linked polymeric polysaccharides having a hydrophobic modifying agent containing at least one first site, which is attached to an oxidized ferulic acid of the polymeric polysaccharide, and at least one second site, which provides desired properties to the polymeric polysaccharide matrix.

Still, the present invention relates to a product being coated with a modified polymeric polysaccharide matrix comprising cross-linked polymeric polysaccharides having a hydrophobic modifying agent containing a first site, which is attached to an oxidized ferulic acid of the polymeric polysaccharide, and a second site, which provides desired properties to the polymeric polysaccharide matrix.

The present invention further relates to a use of a modified polymeric polysaccharide matrix of the invention in thickening agents, hydrogels, films, edible coatings or coatings of packaging materials and to a use of a product of the invention for manufacturing packages of food products, animal feed, cosmetics or electronics.

The benefit of this application is to provide a novel polymeric polysaccharide containing biomaterial applicable for food and cosmetics industry. Coating of biomaterial, such as paper or pasteboard, with the modified polymeric polysaccharide matrix of this invention provides new packaging biomaterial. The aim of using the biobased films and coatings is extending food shelf life, improving quality and usability of a food product or a cosmetic as well as reducing the amount of synthetic packaging materials.

The present invention also enables the use of only single polymeric polysaccharide containing film instead of conventional multilayers of different films. Furthermore, natural solutions of sustainable development are provided.

The methods and means of the invention accomplish new features of biomaterial, including barrier capacities, such as oil, gas, water and water vapour barriers, and therefore, improve the utility of such biomaterials.

### BRIEF DESCRIPTION OF THE DRAWINGS

In the following the invention will be described in greater detail by means of preferred embodiments with reference to the attached drawings, in which

FIG. 1 shows results of a dissolution test of cross-linked pectin films into water. Films cross-linked by laccase dosage of 1-5 nanokatals/g (7% pectin, 60° C.) were insoluble when immersed into water, whereas the reference (no enzyme, i.e. untreated control sample) and the film treated with the low laccase dosage (0.5 nkat/g) were dissolved.

FIGS. 2a-d show images taken after grease resistance test on backsides of the card boards coated with modified pectin. All samples contained 7% pectin, 2% bacterial microcrystalline cellulose (BMCC), 3% Imerol and 35% of glycerol. a) Reference, b) cross-linked with Trametes hirsutalaccase (ThL), c) Reference+DOGA and d) cross-linked and functionalized with DOGA by ThL. Native pectin is a good barrier for grease, but it looses its grease barrier in humid conditions. Additionally, the wetting agent (Imerol) and DOGA destroyed also grease barrier when applied without laccase treatment. Cross-linking with laccase was a necessity to retain grease resistance after functionalization with the hydrophobic component (DOGA) and/or in presence of the wetting agent.

FIG. 3 shows oxygen transmission rates (OTR) (cc/m²/day) of pectin coatings obtained by laccase induced cross-linking and functionalization with DOGA or PROGA. Measurements were performed at RH 80%. For comparison, OTR for the polyethylene coated cardboard (StoraEnso, Cupforma Classic) was ˜4700 cc/m²/day at RH 80%.

FIGS. 4a-b show tensile strength (a) and strain (b) of pectin films cross-linked and functionalized with laccase and DOGA. Gly35′)/0 and TG35% refer to 35% (w/w of pectin) glycerol and glycerol ether 10, respectively. Choice of the plasticizer affected greatly on strength and strain properties of the pectin films. Replacement of glycerol with TG 10 resulted to very strong films. Cross-linked and DOGA modified films that were plasticized with TG 10 had 50% higher tensile strength as compared with corresponding films plasticized with glycerol. Instead, the pectin films plasticized with TG 10 had low strain values.

FIGS. 5a-b show strength properties (a. tensile strength, b. strain) of pectin films reinforced with bacterial microcrystalline cellulose (BMCC) and sugar beet (nano)cellulose (Danicell). CMC refers to carboxy methyl cellulose. Strength properties of pectin films were improvement by supplement of (nano)cellulose. Increasing trend of tensile strength as a function of cellulose charge was detected both for the cross-linked and cross-linked+functionalized films. The highest values were recorded for Danicell at the charge of 2.5%. Flexibility of pectin films was clearly increased by addition of both Danicell and BMCC (5b).

FIG. 6 shows the dissolution of pectin films in water. Pectin crosslinked with APS was insoluble to water.

FIG. 7 shows the solubility of the cross-linked and functionalized pectin films. 1. Sugar beet pectin, 2. Sugar beet pectin+APS, 3. Sugar beet pectin+APS+20 mg/g HexVan and 4. Sugar beet pectin+APS+HexVan 60 mg/g.

### DETAILED DESCRIPTION OF THE INVENTION

### Polymeric Polysaccharides for Modification

It has been found a novel method for modifying biomaterial, which is a natural polymer, specifically polymeric polysaccharide. “Polymeric polysaccharide” refers to material extracted from plant biomass, cellulosic harvest or crop residues, industrial by-product (e.g. from sugar production) or waste. Polymeric polysaccharides modified in the present invention include any polymeric polysaccharides from natural sources. The isolated polymeric polysaccharides used in the present invention may also be further modified by synthetic means. In a preferred embodiment of the invention, the polymeric polysaccharide is a pectin or xylan.

Preferred pectins include but are not limited to pectins of sugar beet, apple pomaces, citrus fruits, potatoes, tomatoes and pears. Sugar beet pectin is a preferred barrier material for the present invention.

Preferred xylans include but are not limited to gramineous xylans of monocots. Arabinoxylan is a preferred barrier material for the present invention.

In the present invention, the polymeric polysaccharide matrix comprises any part or fragment of the polysaccharide, provided that the part or fragment comprises ferulic acid (FA). Indeed, polymeric polysaccharides of the invention (e.g. pectins and/or xylans) are characterized by ferulic acid residues, which act as sites for chemical modification. If the polymeric polysaccharide does not naturally have an FA group or their number needs to be increased, it is possible to graft these groups to the polysaccharide by synthetic means. Chemical formula of a ferulic acid is shown below. “Ferulic acid” refers to (E)-3-(4-hydroxy-3-methoxy-phenyl)prop-2-enoic acid and derivatives thereof.

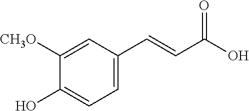

In a preferred embodiment of the invention the polymeric polysaccharide matrix comprises at least one of the following: both a smooth and a hairy region of pectin; a hairy region of pectin; arabinoxylan with ferulic acid residues; and any derivative thereof.

### Modification of Polymeric Polysaccharides

Polymeric polysaccharides are significant constituents in renewable raw materials. Enzymes or chemicals can be used for modification of the polymeric polysaccharides and their technological properties in these materials. Polymeric polysaccharide matrix can also be modified by physical modification, such as irradiation and heat curing.

### Cross-Linking

Covalent cross-linking is a valuable mechanism for increasing the strength and strain of tridimensional polysaccharide networks and providing greater physical integrity in aqueous media. The restrain of cross-linking on the segmental mobility of the polymer makes the diffusion process slower leading to decrease in permeability and solubility of the polymeric polysaccharide matrix to aqueous solvents.

The cross-linked polymeric polysaccharide matrix may also have greater physical integrity in solvents lacking water. For example, these solvents include but are not limited to methanol, ethanol and acetone.

As used herein, the expression “cross-linking” is the formation of intermolecular bonds among the chains of a polymer. Cross-linking occurs between the oxidized ferulic acid components of polymeric polysaccharides.

### By Enzymes

Enzymatic treatments of polymeric polysaccharides can be utilized to form inter- and intramolecular cross-links in polymeric polysaccharides and thus, to improve film properties. Any type of enzyme capable of catalyzing oxidation of phenolic groups may be used in the present invention.

Phenol oxidases using oxygen as an electron acceptor are particularly suitable for enzymatic processes as no separate cofactors needing expensive regeneration, i.e. NAD(P)H/NAD(P) are required in the reactions. These phenol oxidases include e.g. laccase and tyrosinase. They are both copper proteins and can oxidize various phenolic compounds. The substrate specificity of laccases and tyrosinases is partially overlapping.

Tyrosinase catalyses both the o-hydroxylation of monophenols and aromatic amines and the oxidation of o-diphenols to o-quinones or o-aminophenols to o-quinoneimines (Lerch K., 1981. Copper monooxygenases: Tyrosinase and dopamine γ-hydroxylase. In H. Sigel (Ed.), Metal ions biological systems (pp. 143-186). New York, Marcel Dekker). Traditionally tyrosinases can be distinguished from laccases on the basis of substrate specificity and sensitivity to inhibitors. However, the differentiation is nowadays based on structural features. Structurally the major difference between tyrosinases and laccases is that tyrosinase has a binuclear copper site with two type III coppers in its active site, meanwhile laccase has altogether four copper atoms (type I and II coppers, and a pair of type III coppers) in the active site.

Laccases form radicals to polymeric polysaccharides and also to other possible substrates (e.g. phenolic components or small molecules). Therefore the process is more difficult to control than quinone-derived non-radical reactions catalyzed by tyrosinase. Properties and dosage of a laccase preparation and treatment conditions, such as temperature, pH, O₂ concentration, mixing and treatment time, affect on quantity and shelf-life of formed radicals and hence on cross-linking and/or functionalization of polymeric polysaccharide.

Peroxidase, such as horseradish peroxidise, treatment may also be used for polymeric polysaccharide film forming solutions. In case peroxidases are used in the enzymatic reaction, hydrogen peroxide must be present as an oxidizing agent.

In a preferred embodiment of the invention, the cross-linking is carried out by an enzyme catalysed reaction.

In a preferred embodiment of the methods of the invention the enzyme for cross-linking is selected from the group consisting of laccases (EC 1.10. 3.2), catechol oxidases (EC 1.10.3. 1), tyrosinases (EC 1.14. 18. 1), bilirubin oxidases (EC 1.3. 3.5), horseradish peroxidases (EC 1.11. 1.7), manganase peroxidases (EC 1.11.1. 13), lignin peroxidases (EC 1.11. 1.14), hexose oxidases (EC 1.1. 3.5), galactose oxidases (EC 1.1. 3.9) and lipoxygenases (EC 1.13. 11.12). Most preferably the enzyme is a laccase or tyrosinase, preferably laccase. Laccase or tyrosinase can be selected from laccases or tyrosinases obtainable from plants, mammals, and insects or from microbial sources like Agaricus bisporus, Neurospora, Streptomyces, Bacillus, Myrothecium, Mucor, Miriococcum, Aspergillus, Chaetotomastia, Ascovaginospora, Trametes or Trichoderma.

In addition to being obtainable from living organisms, an enzyme used in the present invention can be produced for example by synthetic or recombinant production. Any method known in the art can be used for the production of a suitable enzyme.

### By Chemicals

Polymeric polysaccharides intended for nonfood applications are able to be cross-linked by a broad variety of chemical agents. Bifunctional and multifunctional reagents, such as diisocyanates and carbodiimides, have been used to improve functional properties of films made from keratin, wheat gluten, and zein. Diisocyanates act as lysine targeted cross-linkers, and carbodiimides selectively link carboxylic acid and phenolic groups. Formaldehyde has the broadest reaction specificity, being able to cross-link ferulic acids of polymeric polysaccharides, thus promoting the formation of intra- and intermolecular covalent bonds. Dialdehydes, glutaraldehyde or glyoxal may also be used as cross-linkers in polymeric polysaccharides. Besides aldehydes, other chemical agents, such as epichlorohydrin or sodium dodecyl sulphate, can be used to modify polymeric polysaccharide film properties.

### By Physical Treatment

Phenolic groups can absorb UV radiation and recombine to form covalent cross-links in polymeric polysaccharides.

γ-Irradiation affects polymeric polysaccharides by causing conformational changes, oxidation of phenolic groups, rupture of covalent bonds, and formation of free radicals. Chemical changes in the polymeric polysaccharides caused by γ-irradiation include cross-linking but also fragmentation, aggregation and oxidation. Two hypotheses have been stated to explain the effect on γ-irradiation: (i) a participation of more molecular residues in intermolecular interactions in polymers with different physicochemical properties and (ii) the formation of inter- and/or intramolecular convalent cross-links in the film forming solutions (Ouattara, B. et al. 2002, Radiation Physics and Chemistry, Vol 63 (3-6), p. 821-825).

In addition to radiation, thermal treatments of polymeric polysaccharides may promote formation of intramolecular and intermolecular cross-links.

### Functionalization

Functionalization of polymeric polysaccharides comprises the steps of 1) oxidizing ferulic acids to provide an oxidized material, and 2) contacting the oxidized material with a modifying agent. Thus, functionalization, i.e. adding modifying agents to polymeric polysaccharides, results in modified properties of the biomaterial foreign to the native polymeric polysaccharide. The achieved properties depend on the modifying agent in use.

As used herein, the expression “oxidizing” refers to an oxidoreductase enzyme, chemical or radiation catalysing the formation of a reactive quinone or a radical intermediate. Typical examples of these types of reactions are shown on page 15.

In the first stage of the present functionalizing process, the polymeric polysaccharide matrix is reacted with a substance capable of catalyzing the oxidation of phenolic or similar structural groups, such as ferulic acids, to provide an oxidized polymeric polysaccharide matrix. Typically, the substance is an enzyme and the enzymatic reaction is carried out by contacting the polymeric polysaccharide matrix with an oxidizing agent, which is capable, in the presence of the enzyme, of oxidizing the ferulic acids to provide the oxidized matrix.

Instead of enzymes, the polymeric polysaccharide matrix can be reacted with a chemical oxidizing agent capable of catalyzing the oxidation of ferulic acids to provide the oxidized polymeric polysaccharide matrix.

Oxidizing agents can be oxygen and oxygen-containing gases, such as air, and hydrogen peroxide. Oxygen can be supplied by various means, such as efficient mixing, foaming, air enriched with oxygen or oxygen supplied by enzymatic or chemical means, such as peroxides to the solution.

The chemical oxidizing agent may also be a typical, free radical forming substance, such as Fenton reagent, organic peroxidase, potassium permanganate, ozone and chloride dioxide. Examples of suitable salts are inorganic transition metal salts, specifically salts of sulphuric acid, nitric acid and hydrochloric acid. Strong chemical oxidants, such as alkali metal and ammoniumpersulphates and organic and inorganic peroxides can be used as oxidising agents in the first stage of the present process.

The chemical oxidants capable of oxidation of phenolic groups can be compounds reacting by radical mechanism. The oxidizing agent can also be any oxidizing initiator, i.e. an agent initiating the oxidation.

To provide the oxidized polymeric polysaccharide matrix, the polymeric polysaccharide matrix can also be reacted with a radical forming radiation capable of catalyzing the oxidation of ferulic acids. Radical forming radiation comprises gamma irradiation, electron beam radiation or any high energy radiation capable of forming radicals in polymeric polysaccharide matrixes.

In a preferred embodiment of the invention, oxidation results from a combination of chemical and biochemical treatments.

Generally, the first step of the process lasts for about 0.1 minutes to 24 hours, typically about 1 minute to about 10 hours, depending on the oxidizing substance employed. The treatment time can be, for example, about 5 to 240 minutes, in the case of enzymes.

In a preferred embodiment of the methods, the functionalization involves an enzyme catalysed reaction.

In a preferred embodiment of the methods, the enzyme for functionalization is selected from the group consisting of tyrosinases (EC 1.14. 18. 1), laccases (EC 1.10. 3.2), catechol oxidases (EC 1.10.3. 1), bilirubin oxidases (EC 1.3. 3.5), horseradish peroxidases (EC 1.11. 1.7), manganase peroxidases (EC1. 11.1. 13), lignin peroxidases (EC 1.11. 1.14), hexose oxidases (EC 1.1. 3.5), galactose oxidases (EC 1.1. 3.9) and lipoxygenases (EC 1.13. 11.12). Most preferably the enzyme is selected from the group consisting of tyrosinases and laccases. Laccase is the most preferred enzyme for the functionalization. Laccase can be selected from laccases obtainable from Melanocarpus (EC 1.10.3.2), from Trametes (EC 1.10.3.2), from Pycnoporus (EC 1.10.3.2), from Rhizoctonia (EC 1.10.3.2), from Coprinus (EC 1.10.3.2), from Myceliophtora (EC 1.10.3.2), from Pleurotus (EC 1.10.3.2), from Rhus (EC 1.10.3.2), from Agaricus (EC 1.10.3.2), from Aspergillus (EC 1.10.3.2), from Cerrena (EC 1.10.3.2), from Curvularia (EC 1.10.3.2), from Fusarium (EC 1.10.3.2), from Lentinius (EC 1.10.3.2), from Monocillium (EC 1.10.3.2), from Myceliophtora (EC 1.10.3.2), from Neurospora (EC 1.10.3.2), from Penicillium (EC 1.10.3.2), from Phanerochaete (EC 1.10.3.2), from Phlebia (EC 1.10.3.2), from Podospora (EC 1.10.3.2), from Schizophyllum (EC 1.10.3.2), from Sporotrichum (EC 1.10.3.2), from Stagonospora (EC 1.10.3.2) from Chaetomium (EC 1.10.3.2), from Bacillus (EC 1.10.3.2), from Azospirillum (EC 1.10.3.2) and from Trichoderma (EC 1.10.3.2). In addition to being obtainable from living organisms, the enzyme can be produced for example by synthetic or recombinant production. Any method known in the art can be used for the production of a suitable enzyme.

Examples of specified structures of typical laccase and tyrosinase substrates are presented below.

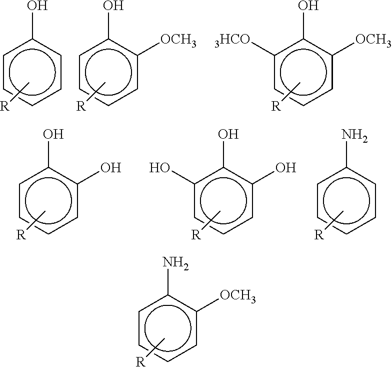

Furthermore, examples of laccase and tyrosinase reactions are shown below.

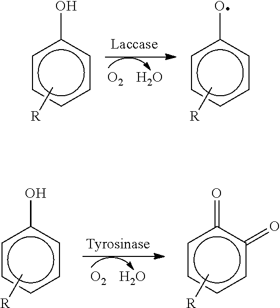

In the second step of the functionalizing process, a modifying agent is bonded to the oxidized ferulic acids of the matrix. Such a modifying agent typically exhibits at least one first site, which is compatible with the polymeric polysaccharide matrix, and optionally at least one second site, as will be explained in more detail below.

In the second stage of the present process, the modifying agent is able to react with the oxidized material.

As used herein, the expression “first site” of the modifying agent refers to a site, which is reactive with the oxidized groups of the polymeric polysaccharides. The modifying agent can have a plurality of first functional sites (see WO2005/061790). Typically, there are 1 to 3 first functional groups, although the bonding of the modifying agent to the polymeric polysaccharide matrix would appear to take place mainly through one functional group at the time. One functional site or component may cause several properties to the polymeric polysaccharide matrix.

The modifying agent can further have a second functional site or sites, which comprise(s) either functionalities, which render the bonded agent and the polymeric polysaccharide substrate to which it is bonded specific properties directly derivable from the second functionality, or functionalities, which are suitable for attaching a functional agent. As used herein, the expression “second site” of the modifying agent refers to a site, which provides desired properties to the polymeric polysaccharide matrix.

The functional sites or groups of the modifying agents can be identical or different. The functional groups can be any of, for example, typical chemical reactive groups, such as hydroxyl (including phenolic hydroxy groups), carboxy, anhydride, aldehyde, ketone, amino, amine, amide, imine, imidine and derivatives and salts thereof, to mention some examples. Also electronegative bonds, such as double bonds, oxo or azobridges, can provide for bonding to the oxidized residues. Any group capable of achieving a bond to a functional site is included. The bond can be based on ionic or covalent bonding or hydrogen bonding. According to a preferred embodiment of the invention, the modifying agent and polymeric polysaccharides form covalent bonds.

The groups of the modifying agents capable of carrying or capable of being modified for carrying any properties may provide properties, such as a negative or positive charge, antibacterial, antifungal or antimicrobial effect, heatproof, flame-retardant or UV-resistant, colour, or any oxygen/gas barrier properties.

In the modifying agents, a hydrocarbon residue, to which the functional site or sites is attached, can be linear or branched aliphatic, cycloaliphatic, heteroaliphatic, aromatic or heteroaromatic. The hydrocarbon residue can be saturated or unsaturated.

In the invention, the modifying agent is hydrophobic. Examples of preferred modifying agents are compounds, which comprise a hydrophobic hydrocarbon tail. Such compounds are exemplified by methoxy- and dimethoxyphenols, such as eugenol, isoeugenol, vanilic acid, ferulic acid and their alkyl derivatives, and derivatives of phenolic or aniline type compounds such as gallate/gallic acid, 3,4-dihydroxy benzoic acid, caffeic acid, vanilyl amine, tyramine, L-Dopa and tyrosine to name a few examples.

In a further preferred embodiment of the invention, the modifying agent has a hydrocarbon tail, which contains a minimum of two, preferably at least three carbon atoms, and a maximum of up to 30 carbon atoms, in particular up to 24 carbon atoms. Such chains can be the residues of fatty acids bonded to the core of the modifying agent.

According to a preferred embodiment of the invention, the modifying agent is selected from the group consisting of phenols, methoxyphenols, aniline derivatives, primary amines, thiols, alkyl derivatives of gallate gallic acid, such as dodecyl gallate (DOGA), octyl gallate (OGA) and propyl gallate (PROGA), and derivatives or structural analogues thereof. These agents are able to increase hydrophobic properties of the biomatrix.

In a preferred embodiment of the methods, the modifying agent is DOGA, OGA or PROGA, most preferably DOGA. DOGA is an ester of dodecanol and gallic acid, a small molecule, which is an acceptable additive in food products and cosmetics. The structures of DOGA and PROGA are presented below.

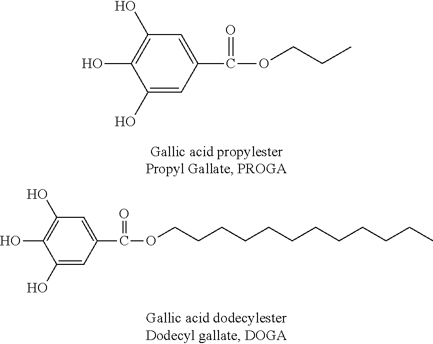

In one embodiment of the invention, the modifying agent of the functionalization step is activated with an oxidizing agent.

The first and second steps of the functionalization can be carried out sequentially or simultaneously. According to a particularly preferred embodiment, the first and the second stages of the functionalization process are carried out in the same reaction medium, without separating the polymeric polysaccharide matrix after the oxidation step. The conditions (consistency, temperature, pH, pressure) can, though, even in this embodiment be different during the various processing stages.

### Combining Cross-Linking and Functionalization

Both cross-linking and functionalization of polymeric polysaccharides occur through ferulic acids.

Cross-linking and functionalizing of polymeric polysaccharides are sequential or simultaneous reactions. The method steps can be carried out sequentially by first cross-linking and then functionalizing polymeric polysaccharides or first functionalizing and then cross-linking polymeric polysaccharides of the biomaterial. The sequence of events depends on the enzyme/enzymes as well as the reaction conditions used.

In one preferred embodiment of the invention, polymeric polysaccharides are first functionalized and then cross-linked. In another preferred embodiment of the invention, polymeric polysaccharides are first cross-linked and then functionalized.

In one preferred embodiment of the invention, cross-linking and functionalizing are carried out simultaneously. In one specific embodiment of the invention, only one enzyme is used in cross-linking and functionalizing. A preferred enzyme for these reactions is a laccase.

The method of the invention comprising the cross-linking and the functionalizing can be carried out enzymatically, chemically or by physical treatment. In one embodiment of the invention cross-linking and/or functionalizing is an enzyme-catalysed reaction. According to a specific embodiment, at least one enzyme, such as laccase, or at least two different enzymes, such as 1) laccase and 2) tyrosinase, are used in cross-linking and functionalizing, respectively. According to one embodiment of the invention, at least one enzyme or at least two different enzymes are used in cross-linking or functionalizing.

The conditions (for example consistency, temperature, pH, pressure) can be different during the various processing steps. In a preferred embodiment of the invention, the enzyme dosage is from 0.1 to 100 000 nkat/g of dry matter, preferably 1-1000 nkat/g of dry matter. In another preferred embodiment, the enzyme dosage is employed in an amount of 0.0001 to 10 mg enzyme protein/g of dry matter.

In one embodiment of the invention cross-linking and/or functionalization of polymeric polysaccharides is carried out as a chemically catalysed reaction. In one embodiment of the invention the method is carried out chemically or by radiation at least in part.

Both reactions of the methods can be carried out in an aqueous or solid phase at a consistency of 1 to 95% by weight of the polymeric polysaccharide containing material. Alternatively, one of the reactions can be carried out in an aqueous phase and the other one in a solid phase.

In one preferred embodiment of the invention, the reactions are carried out at temperature 2-100° C., more preferably at temperatures 20-70° C.

In a preferred embodiment of the invention the modified polymeric polysaccharide matrix is obtainable by the method of the invention. Furthermore, in a preferred embodiment of the invention the product being coated with a modified polymeric polysaccharide matrix is obtainable by the method of the invention.

As used herein the expression “product being coated with a modified polymeric polysaccharide matrix” refers to any product, which has been coated. For example the product can be selected from a group consisting of synthetic plastics, fibre comprising materials or products, any unmodified biobased polymer material, domestic chemicals, cosmetic products or edible products.

In a preferred embodiment of the invention the modified polymeric polysaccharide matrix or the product being coated with a modified polymeric polysaccharide matrix has improved barrier properties to one or more of the substances selected from the group consisting of gases, water vapour, aroma compounds and greases compared to unmodified polymeric polysaccharide matrix or the product, respectively. In another preferred embodiment of the invention the modified polymeric polysaccharide matrix or the product being coated with a modified polymeric polysaccharide matrix has improved maintenance of the oxygen barrier properties in high relative humidities. In a preferred embodiment of the invention the modified polymeric polysaccharide matrix or the product being coated with a modified polymeric polysaccharide matrix is impermeable to water vapour. In a preferred embodiment of the invention the modified polymeric polysaccharide matrix or the product being coated with a modified polymeric polysaccharide matrix has improved mechanical properties selected from the group consisting of elasticity, strength and strain compared to unmodified polymeric polysaccharide matrix or the product, respectively. Any known methods can be used in measuring or studying the barrier or mechanical properties of materials or products. Some of those methods are described in the examples of this application.

### Processes for Biopackaging

Two general processes for biopackaging formation can be distinguished: the wet and the dry process. The wet process can be based on a film-forming (water) dispersion of biopolymers, spraying of the (water) dispersion of biopolymers or extrusion of the (water) dispersion of biopolymers. The dry process is based on the thermoplastic properties of biopolymers heated above their glass transition temperature under low water content conditions.

Many processing procedures have been used to form coatings and films, such as dipping, spraying, foaming, fluidization, enrobing, casting and extrusion. All of them could be employed for the polymeric polysaccharide films. In a preferred embodiment of the invention, coating methods, which are applicable as unit operations in continuous, reel-to-reel manufacturing processes are preferred. These methods include spraying, curtain coating, dispersion coating, printing (e.g. ink jet printing, screen printing, flexo printing, gravure printing) and combinations thereof.

In a preferred embodiment of the invention, cross-linking of polymeric polysaccharides is carried out by spraying the enzyme on polymeric polysaccharide coated cardboard or by dispersion coating.

Any additives, such as plasticizers, which improve the properties of polymeric polysaccharide matrixes or products, may be added during processing, modification or coating procedures of the present invention. In a preferred embodiment of the invention, a plasticizer or plasticizers selected from a group consisting of glycerol ether (e.g. TG-10), glycerol and sorbitol is/are used in the methods of the invention.

### Utility

The biomaterial of the invention is useful both in food and non-food applications. The method of the invention can be used in treating biopolymer, which contains polymeric polysaccharides.

The biomaterial product of the invention has properties useful for packaging of many different food products, because it functions as an efficient barrier of oxygen, water vapour and oil. The modified polymeric polysaccharide of the invention can be used for coating processes, such as paper or cardboard coatings. The novel biopolymer assorts exquisitely for packaging of any dried product, animal food or fast food, such as cereals, hamburgers or cookies, as well as pharmaceuticals or cosmetics.

The products of the present invention can furthermore be utilized as distinct films, thickening agents or hydrogels.

The following examples are given for further illustration of the invention.

It will be obvious to a person skilled in the art that, as the technology advances, the inventive concept can be implemented in various ways. The invention and its embodiments are not limited to the examples described below but may vary within the scope of the claims.

### EXAMPLE 1 PRODUCTION AND PURIFICATION OF THE ENZYMES

### Laccase

The Trametes hirsuta-laccase (ThL) was purified as follows. The culture filtrate from T. hirsuta strain VTT D-443 was concentrated by ultrafiltration (PCl, 25-kDA cut off). Salts were removed from the concentrate and the buffer changed to 15 mM acetate buffer pH 5.0 by gel filtration (Sephadex G 25; h=57 cm, V=18 l). The active fractions were pooled and the solution was concentrated again by ultrafiltration (PCl, 25-kDa cut off). The sample was applied to a DEAE Sepharose Fast Flow anion exchange column (h=29 cm, V=9 l), which was equilibrated with 15 mM sodium acetate pH 5.0. The proteins were eluted with a linear 0-200 mM NaCl gradient. Laccase eluted at 120-150 mM NaCl concentration. Positive fractions were pooled and Na₂SO₄ was added to the sample to final concentration of 1 M. The sample was applied to a Phenyl Sepharose Fast Folw hydrophobic interaction column (h=20 cm, V=400 ml) equilibrated with 20 mM citrate buffer pH 5.0 containing 1 M. The proteins were eluted with a linear decreasing Na2SO4 gradient (1000-0 mM) Na₂SO₄. Laccase eluted at 20 mM Na₂SO₄ salt concentration. The purest fractions were pooled and concentrated (Millipore; PM10 membrane). All chromatographic resins were supplied by Pharmacia.

### Tyrosinase

A tyrosinase from the filamentous fungus Trichoderma reesei was over-expressed under a strong cbh1 promoter in its native host. The tyrosinase gene tyr2 of T. reesei encoded a protein with a signal sequence, and the protein was observed to be secreted in a high titer to the culture supernatant in laboratory-scale batch fermentation (20 L). T. reesei tyrosinase was purified with a three-step purification procedure, consisting of desalting by gel filtration chromatography, cation exchange chromatography and gel filtration chromatography. The purified tyrosinase protein had a molecular mass of 43.2 kDa. T. reesei tyrosinase showed the highest activity and stability within a neutral and alkaline pH range, having an optimum at pH 9. T. reesei tyrosinase retained its activity well at 20-30° C., whereas at higher temperatures the enzyme started to lose its activity relatively quickly. The pl of T. reesei tyrosinase was around 9.5. T. reesei tyrosinase was active on both L-tyrosine and L-dopa, and it showed broad substrate specificity.

### EXAMPLE 2. ENZYMATIC CROSS-LINKING OF SUGAR BEET PECTIN

Sugar beet pectin was obtained from Danisco Sugar A/S. Some properties of the sugar beet pectin are shown in Table 1.

TABLE 1
General properties and quality of the sugar beet pectin

| General properties | |
|---|---|
| Dry substance | 95.2% |
| pH | 3.8 |
| Molecular weight | 31 500 g/mol |
| Quality | |
| Purity | 99.3% |
| Galacturonic acid | 72.4% |
| Acetic acid | 5.3% |
| Degree of methylation | 61.8% |
| Degree of acetylation | 23.4% |

The pectin solution (7% w/w) for stand alone films or coatings was prepared as follows: Pectin was dispersed into deionised water under mixing with a magnetic stirrer and glycerol (33.5%) was added. Glycerol was used as the plasticiser. The pectin solution was slightly heated up and pH was adjusted to 4.5 with 1 M NaOH. Degassing of the pectin solution was carried out with an ultrasonic bath prior to use for preparation of stand alone films or coating on card board in order to avoid gas bubbles and pin holes.

Cross-linking of pectin was catalysed by laccase. The treatments of pectin solution (7% w/w, pH ˜4.5) with Trametes hirsuta laccase were carried out at 60° C. The dosage of laccase varied between 0.5 and 5 nkat/g of pectin. After addition of laccase the pectin solution was mixed thoroughly and poured on petri dishes to obtain stand alone films. The petri dishes were let to dry at room temperature (20° C., RH 50%) for 2 days.

The degree and rate of cross-linking of pectin by laccase was dependent on the enzyme charge and temperature. In the standardized procedure (7% pectin, 60°) the laccase dosage of 1-3 nanokatals/g of pectin was enough to have cross-linked and even stand alone films (FIG. 1). The crosslinked films were insoluble to water when immersed into water. This was not the case with the untreated control sample (no enzyme), as can be observed in FIG. 1 and Table 2.

It was concluded that the pectin films cross-linked by laccase (dosage 1-5 nanokatals/g) remained intact, whereas the reference (no enzyme) and the film treated with the low laccase dosage (0.5 nkat/g) were dissolved (FIG. 1 and Table 2).

TABLE 2
Dissolution of pectin films cross-linked with laccase

| Sample | Dissolution |
|---|---|
| Reference (no enzyme) | + |
| Laccase 0.5 nkat/g | + |
| Laccase 1 nkat/g | − |
| Laccase 3 nkat/g | − |
| Laccase 5 nkat/g | − |

### EXAMPLE 3. CROSS-LINKING AND FUNCTIONALIZATION OF SUGAR BEET PECTIN

Pectin solution was prepared as described in Example 2. Motivation for simultaneous cross-linking and functionalization of sugar beet pectin was to obtain insoluble pectin matrix with less hydrophilic nature in a one step treatment. Aquatic dispersion of dodecyl gallate (DOGA, Merck), octyl gallate (OGA, Lancaster), or aquatic solution of propyl gallate (PROGA, Acros) was used as hydrophobic agents. DOGA, OGA, or PROGA were added to the reaction mixture at the concentration of 10 or 20 mg/g of pectin.

Aqueous dispersions of DOGA and OGA were prepared as follows: 0.821 g DOGA (dodecyl 3,4,5-trihydroxybenzoate, Merck) or OGA (3,4,5-trihydroxybenzoic acid octyl ester), 0.08 fennodispo A41, 20 ml acetone and 20 ml distilled water were mixed in a beaker and heated until acetone was evaporated. After that a mixture containing 0.04g lecithin (L-α-P- from egg yolk, Fluka) in approximately 40 ml of water (60° C.) was added and the final volume was adjusted to 100 ml. The aqueous solution of PROGA was obtained by dissolution directly into distilled water.

A commercial wetting agent Imerol (Clariant) was used (final concentration 3%) in order to improve dispersion of pectin on Petri dishes (stand alone films) and cardboard (coating experiments). Different reactants were mixed (60° C.) in the following order: pectin+Imerol+DOGA (or OGA or PROGA)+laccase having a short mixing period (1-2 min) between each addition. The dosage of laccase was varied between 1 and 10 nanokatals/g of pectin. In the case of stand alone films the pectin solution was poured after mixing to Petri dishes and left dry (20° C., relative humidity (RH) 50%), whereas in the case of hand coating the reaction time was 20 min prior to drying of coated card board at 80° C. for 20 min.

The coatings were applied with using a K Hand Coater (RK Print Coat Instruments LtD. The thickness of the wet pectin layer was 100 μm. Cupforma Classic cardboard (Stora Enso) was used as the basis substrate for coating trials.

The contact angle measurement was carried out on card boards using the contact angle meter CAM 200 device. The contact angle was recorded after 2 seconds after application at room temperature and RH 50%. The results are shown in Table 3.

TABLE 3
Contact angle of the pectin coated card boards

| Sample/Treatment | Contact angle, degrees (°) |
|---|---|
| Pectin (ref) | 74.1 |
| Pectin + laccase | 76.0 |
| Pectin + PROGA | 76.9 |
| Pectin + PROGA + laccase | 82.5 |
| Pectin (Imerol) | 44.1 |
| Pectin + laccase | 43.5 |
| Pectin + OGA | 81.2 |
| Pectin + OGA | 81.1 |
| Pectin + DOGA | 84.0 |
| Pectin + DOGA + laccase | 90.6 |

The contact angle of native pectin (no treatment) without and with the wetting agent (Imerol) was 74.1 and 44.1 degrees, respectively. Addition of DOGA or PROGA increased the contact angles, but the highest contact angle values with both hydrophobic agents were obtained when laccase was included in the treatment. The highest contact angle of 90.6 degrees was recorded with DOGA together with the laccase treatment. The results proved that less hydrophilic pectin coating could be obtained by laccase catalysed crosslinking and functionalization with DOGA or PROGA. After cross-linking with laccase DOGA could not be extracted from the solidified film matrix by acetone indicating of chemical bonding between pectin and DOGA or physical entrapment of DOGA inside pectin matrix. From the reference sample (no laccase) DOGA could be extracted quantitatively with acetone.

### EXAMPLE 4. GREASE RESISTANCE OF PECTIN COATING

The oil/grease resistance of the modified pectin films was studied with the modified Tappi T 507 procedure (similar to ASTM F 119). Briefly, a filter paper saturated with coloured olive oil and smaller than the sample was placed on the coating side of the board and a white blotting paper was placed below the sample. The sample package was placed between aluminium foils and up to ten sample packages were piled up between smooth metal plates. A weight of 1 kg was placed on top of the pile. The package was then placed on an oven (T=60° C., t=4 h). The front and back sides of the test samples were photographed after test. The penetration of the coloured oil through the coating was visually investigated from the backside of the board.

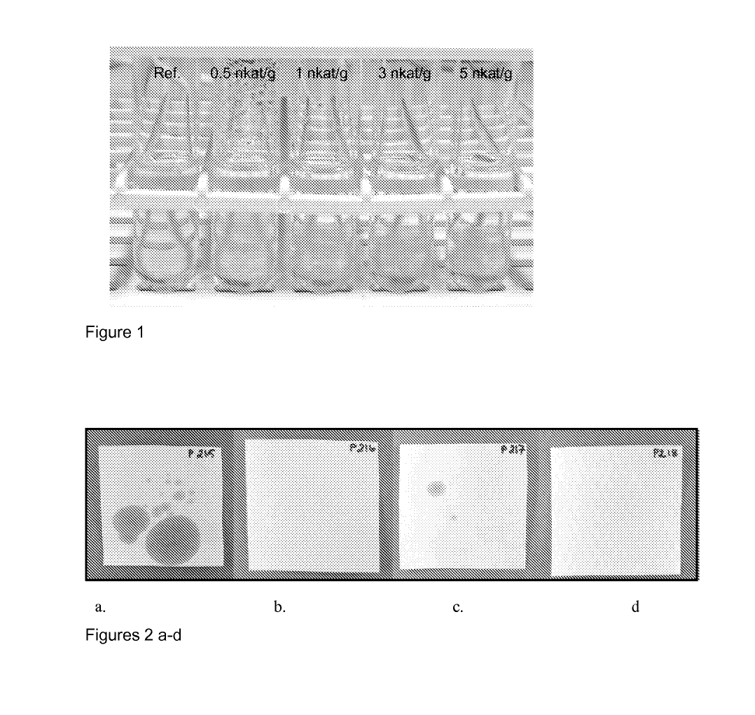

After modification, as described in Example 3, the pectin solutions were hand coated on card board (StoraEnso, Cupforma Classic) using wet thickness of 200 μm (17-18 g/m²). When DOGA was used as the modifying component the wetting agent (Imerol) was added to the pectin solution in order to enhance proper spreading of pectin on card board. The laccase dosage varied between 1 and 10 nkat/g depending on the composition of the reaction mixture and the reaction time. The charges of DOGA and PROGA were 10 and 20 mg/g of pectin, respectively. After drying the card boards were ready for grease resistance testing. The results of the test were evaluated visually and imaged by a digital camera from back side of the card board. The results are shown in FIGS. 2a-d and Table 4.

The images taken after the grease resistance test on backsides of the card boards coated with modified pectin are shown in FIGS. 2a-d. All samples contained 7% pectin, 2% bacterial microcrystalline cellulose (BMCC), 3% Imerol and 35% of glycerol. a) Reference, b) cross-linked with Trametes hirsuta-laccase (ThL), c) Reference+DOGA and d) cross-linked and functionalized with DOGA by ThL.

Native pectin is a good barrier for grease, but it looses its grease barrier in humid conditions. Additionally, the wetting agent (Imerol) and DOGA destroyed also grease barrier when applied without laccase treatment. Cross-linking with laccase was a necessity to retain grease resistance after functionalization with the hydrophobic component (DOGA) and/or in presence of the wetting agent.

TABLE 4
A summary on grease resistance of card boards coated with
pectin modified with DOGA or PROGA

| Sample/Treatment | Grease resistance |
|---|---|
| Pectin | (+) |
| Pectin + ThL | + |
| Pectin + Imerol | − |
| Pectin + Imerol + ThL | + |
| Pectin + DOGA + Imerol | − |
| Pectin + DOGA + Imerol + ThL | + |
| Pectin + PROGA | (+) |
| Pectin + PROGA + ThL | + |

(+), dry conditions
ThL refers to Trametes hirsuta-laccase

### EXAMPLE 5. OXYGEN BARRIER PROPERTIES OF CROSS-LINKED AND FUNCTIONALIZED PECTIN FILMS

Cross-linking and functionalization of sugar beet pectin and coating of card boards were carried out as described in Example 4. A wet layer thickness of 200 μm was typically applied on card boards corresponding to a loading of 17-18 g of pectin per square metre. Both DOGA and PROGA were used as the modifying compounds. A wetting agent (Imerol) was included in the pectin solution when DOGA was used. The OTR values were routinely recorded at RH 80%.

The oxygen barrier properties (OTR) of card boards coated with pectin were analysed with Model 8001 Oxygen Permeation Analyser (Systech Instruments Ltd., UK) or Ox-tran 2/20 Oxygen Transmission Rate System (Mocon, Modern Controls Inc., USA) at different relative humidities (RH) using the methods described in standards ASTM D3985 and F1927. The surface area of the samples was 5 cm².

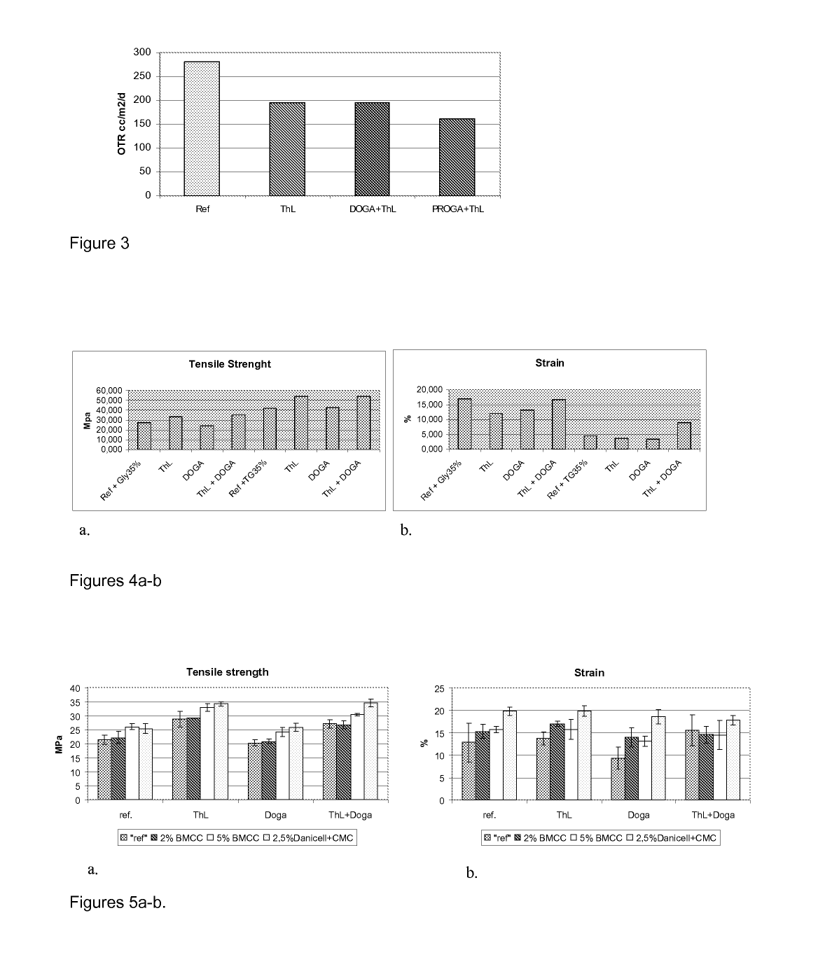

Likewise with other biopolymers the gas permeability of pectin increased with increasing relative humidity. Cross-linking with laccase decreased that tendency and improved O₂ barrier properties of pectin (at RH 80%) as compared with native pectin (FIG. 3). The permeability for oxygen was further decreased by functionalization with PROGA but not as much with DOGA. It was detected that increasing dosage of PROGA had a positive effect on OTR. At a PROGA dosage of 20 mg/g the oxygen permeability at RH 80% decreased >40% as compared with the native pectin.

### EXAMPLE 6. STRENGTH PROPERTIES OF PECTIN FILMS

Polysaccharide-based films are commonly plasticized with polyols, e.g., glycerol or sorbitol, to increase their flexibility. The performance of glycerol and glycerol ether 10 (TG-10) as plasticizers was compared in a pectin matrix at a plasticizer dosage of 35% (w/w of pectin). Stand alone films were prepared from cross-linked (ThL)+functionalized pectin (ThL+DOGA) as described in Example 2. The charges of laccase and DOGA were 1 nkat/g and 10 mg/g, respectively. Dried films were analysed for tensile strength and strain.

The mechanical properties (tensile strength, strain) of the pectin films were analyzed with Texture Analyzer (Stable Micro Systems, UK). The samples (1 cm×6 cm×˜50-100 μm) were cut from cast stand-alone films. The thicknesses of the samples were measured with a micrometer screw. The samples were air-conditioned in controlled conditions (23° C., 50% RH) for at least 24 hours before the measurement. From two to five parallel measurements were made for each sample. The speed used in tensile tests was 1 mm/s. The results are shown in FIGS. 4a and b.

The choice of the plasticizer affected greatly on strength and strain properties of the pectin films. The replacement of glycerol with TG 10 resulted to very strong films. The cross-linked and DOGA modified films that were plasticized with TG 10 had 50% higher tensile strength as compared with corresponding films plasticized with glycerol. Instead, the pectin films plasticized with TG 10 had low strain values.

### EXAMPLE 7. MODIFICATION OF PECTIN MATRIX WITH ADDITIVES

Pectin is a hygroscopic polymer. Enzyme-aided functionalization of pectin decreased the hydrophilic nature of pectin films as concluded in Example 3. Another way to affect on strength and water absorptive properties of pectin is to modify pectin matrix after cross-linking with additives. Bacterial microcrystalline cellulose (BMCC) and sugar beet (nano)cellulose (Danicell) were used as examples of suitable organic components to modify pectin. Danicell preparation was supplemented with carboxy methyl cellulose (CMC) (30% w/w) in order to improve its re-dispersion into water.

During preparation of the pectin solution BMCC and Danicell were added at charges of 2 and 5% for BMCC and at a charge of 2.5% for Danicell. Stand alone films were prepared after cross-linking and cross-linking+functionalization with DOGA as described in Example 4. The films were analysed for strength properties as described in Example 6. The results are summarized in FIG. 5a and b.

The strength properties of the pectin films were improvement by the supplement of (nano)cellulose. An increasing trend of tensile strength as a function of cellulose charge was detected both for the cross-linked and crosslinked+functionalized films. The highest values were recorded for Danicell at the charge of 2.5%, which might be due to CMC giving rise to enhanced adhesion and compatibility within pectin matrix. Surprisingly, the flexibility of pectin films was clearly increased by addition of both Danicell and BMCC (FIG. 5b). The results showed that brittleness of pectin films could be decreased by addition of cellulose nanostructures.

### EXAMPLE 8. CHEMICAL CROSS-LINKING OF SUGAR BEET PECTIN

Ammonium persulphate, APS (NH₄)₂S₂O₈, (Degussa) was used for chemical cross-linking of sugar beet pectin. A 40% solution of ammonium persulphate was prepared in distilled water. The pectin solution was prepared as described in Example 2. In order to cross-link pectin 260 μl of 40% (NH₄)₂S₂O₈ was added to 10.5 g of pectin solution containing 0.735 g of pectin. The mixture was stirred thoroughly to start the reaction. The mixture was kept at room temperature (20° C.) for 15 min and thereafter the mixture was poured to a petri dish to obtain a stand alone film. A reference film was correspondingly prepared but omitting APS. Petri dishes were kept at room temperature (20° C., RH 30%) for 2 days.

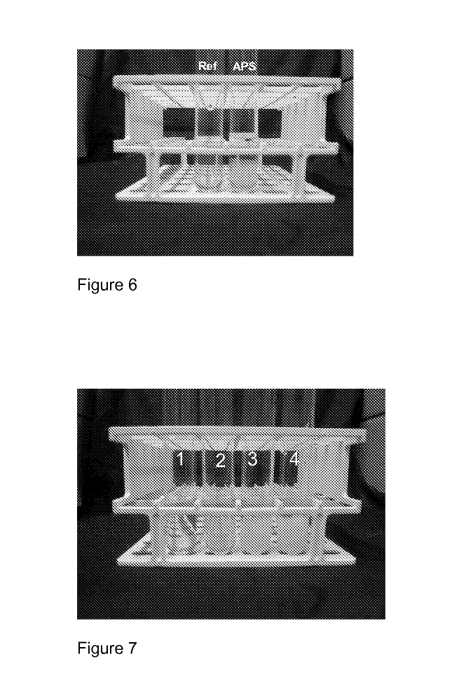

Cross-linking of pectin was verified by immersing small pieces of pectin films into distilled water. The reference film omitting APS was dissolved into water within 5 min whereas the film cross-linked with APS remained insoluble (FIG. 6).

### EXAMPLE 9. CHEMICAL CROSS-LINKING AND FUNCTIONALIZATION OF SUGAR BEET PECTIN

Cross-linking and functionalization of sugar beet pectin was carried out by APS using hexyl vanillate as the modifying component. Hexyl vanillate was synthesised as follows:

The synthesis of vanillic acid hexyl ester was carried by applying method described in U.S. Pat. No. 5,686,406. 40 g of dried vanillic acid (0.24 mole) and 34 g of dry n-hexanol (0.40) mole were added to 125 ml of toluene in a reactor equipped with a Dean-Stark condenser. 5% (3.5 g) of p-toluene sulfonic acid was added as the acidic catalyst. The mixture was refluxed for 24 hours. The elimination of water was observed during period of 12 h. Observed 4 ml yield of water equals well to the theoretical amount of 4.3 ml of water.

After cooling to room temperature, the organic phase was washed with a saturated sodium bicarbonate solution until the pH was neutral. The organic phase was then washed with water before drying over anhydrous sodium sulphate. The toluene and a portion of the n-hexanol were evaporated off under reduced pressure: 130-160° C/0.3 bar. The crude ester was distilled at 170-190° C. under a reduced pressure of 0.05 bar to provide 28 g n-hexyl vanillate having a purity of 97% (NMR) at an overall yield of 46%. Chemical formula of the target substrate: Hexyl-4-hydroxy-3methoxy-benzoate [CAS 84375-71-3]:

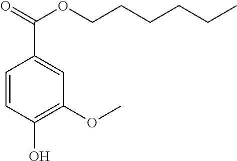

Cross-linking with APS was carried out as described in Example 8. After addition of APS to the pectin solution (260 μl of 40% (NH₄)₂S₂O₈ to 0.735 g of pectin) hexyl vanillate was added to the mixture at dosages of 20 and 60 mg/g (pectin). The mixtures were mixed thoroughly, left to react for 15 min at room temperature (20° C.) and poured onto petri dishes. The petri dishes were kept open at room temperature (20° C., RH 30%, 2 days) and thereafter the pectin films were tested for solubility in water.

The results are shown in FIG. 7. Sugar beet pectin cross-linked with APS appeared as slightly swollen flakes whereas the samples including also hexyl vanillate (HexVan) retained their original film structure and integrity. The pectin film which was not cross-linked with APS dissolved immediately into water.

1. A method of modifying a polymeric polysaccharide matrix, said method comprising
cross-linking polymeric polysaccharides in the matrix, and
functionalizing the polymeric polysaccharides by oxidizing ferulic acids of the polymeric polysaccharides, and contacting the oxidized polymeric polysaccharides with a hydrophobic modifying agent containing at least one first site, which is reactive with the oxidized ferulic acids, and at least one second site, which provides desired properties to the polymeric polysaccharide matrix,
whereby a modified polymeric polysaccharide matrix is obtained.

2. A method of coating a product, said method comprising
providing a polymeric polysaccharide matrix,
cross-linking polymeric polysaccharides in the matrix,
functionalizing the polymeric polysaccharides by oxidizing ferulic acids of the polymeric polysaccharides, and contacting the oxidized polymeric polysaccharides with a hydrophobic modifying agent containing at least one first site, which is reactive with the oxidized ferulic acids, and at least one second site, which provides desired properties to the polymeric polysaccharide matrix to obtain a modified polymeric polysaccharide matrix, and
coating the product with the modified polymeric polysaccharide matrix.

3. A method according to 1 or 2, characterized in that the polymeric polysaccharide matrix comprises at least one of the following: both a smooth and a hairy region of pectin; a hairy region of pectin; arabinoxylan with ferulic acid residues; and any derivative thereof.

4. A method according to any claim 1, characterized in that the cross-linking is carried out by an enzyme catalysed reaction.

5. A method according to claim 4, characterized in that the enzyme for cross-linking is selected from the group consisting of laccases (EC 1.10. 3.2), catechol oxidases (EC 1.10.3. 1), tyrosinases (EC 1.14. 18. 1), bilirubin oxidases (EC 1.3. 3.5), horseradish peroxidases (EC 1.11. 1.7), manganase peroxidases (EC 1.11.1. 13), lignin peroxidases (EC 1.11. 1.14), hexose oxidases (EC 1.1. 3.5), galactose oxidases (EC 1.1. 3.9) and lipoxygenases (EC 1.13. 11.12).

6. A method according to claim 1 or 2, characterized in that the functionalization is carried out by an enzyme catalysed reaction.

7. A method according to claim 6, characterized in that the enzyme for functionalization is selected from the group consisting of tyrosinases (EC 1.14. 18. 1), laccases (EC 1.10. 3.2), catechol oxidases (EC 1.10.3. 1), bilirubin oxidases (EC 1.3. 3.5), horseradish peroxidase (EC 1.11. 1.7), manganase peroxidase (EC1. 11.1. 13), lignin peroxidase (EC 1.11. 1.14), hexose oxidase (EC 1.1. 3.5), galactose oxidase (EC 1.1. 3.9) and lipoxygenases (EC 1.13. 11.12).

8. A method according to claim 1, characterized in that cross-linking and functionalization of polymeric polysaccharides are carried out as sequential or simultaneous reactions.

9. A method according to claim 1, characterized in that cross-linking and/or functionalization of polymeric polysaccharides is carried out as a chemically catalysed reaction.

10. A method according to claim 1, characterized in that both chemically and biochemically catalysed reactions are used.

11. A method according to claim 1, characterized in that cross-linking of polymeric polysaccharides is carried out by spraying the enzyme on polymeric polysaccharide coated cardboard or by dispersion coating.

12. A method according to claim 1, characterized in that the modifying agent has a hydrocarbon tail which contains a minimum of two, preferably at least three carbon atoms, and a maximum of up to 30 carbon atoms, in particular up to 24 carbon atoms.

13. A method according to claim 1, characterized in that the modifying agent is selected from the group consisting of phenols, methoxyphenols, aniline derivates, primary amines, thiols, alkyl derivatives of gallate gallic acid, such as dodecyl gallate (DOGA), odecyl gallate (OGA) and propyl gallate (PROGA), and derivatives or structural analogues thereof.

14. A method according to claim 1, characterized in preparing the modified polymeric polysaccharide matrix or the product being coated with a modified polymeric polysaccharide matrix that has improved barrier properties to one or more of the substances selected from the group consisting of gases, water vapour, aroma compounds and greases compared to unmodified polymeric polysaccharide matrix or product, respectively.

15. A method according to claim 1, characterized in preparing the modified polymeric polysaccharide matrix or the product being coated with a modified polymeric polysaccharide matrix that has improved maintenance of the oxygen barrier properties in high relative humidity.

16. A method according to claim 1, characterized in that the modified polymeric polysaccharide matrix or the product being coated with a modified polymeric polysaccharide matrix has improved mechanical properties selected from the group consisting of elasticity, strength and strain compared to unmodified polymeric polysaccharides or products.

17. A method according to claim 1, characterized in that the modified polymeric polysaccharide matrix or the product being coated with a modified polymeric polysaccharide matrix is impermeable to water vapour.

18. A method according to claim 1, characterized in that a plasticizer or plasticizers selected from a group consisting of glycerol ether, glycerol and sorbitol is/are used in the method.

19. A modified polymeric polysaccharide matrix comprising cross-linked polymeric polysaccharides having a modifying agent containing at least one first site, which is attached to an oxidized ferulic acid of the polymeric polysaccharide, and at least one second site, which provides desired properties to the polymeric polysaccharide matrix.

20. A modified polymeric polysaccharide matrix comprising cross-linked polymeric polysaccharides having a modifying agent containing at least one first site, which is attached to an oxidized ferulic acid of the polymeric polysaccharide, and at least one second site, which provides desired properties to the polymeric polysaccharide matrix, characterized by being obtainable by the method of claim 1.

21. A product being coated with a modified polymeric polysaccharide matrix comprising cross-linked polymeric polysaccharides having a modifying agent containing a first site, which is attached to an oxidized ferulic acid of the polymeric polysaccharide, and a second site, which provides desired properties to the polymeric polysaccharide matrix.

22. A product being coated with a modified polymeric polysaccharide matrix comprising cross-linked polymeric polysaccharides having a modifying agent containing a first site, which is attached to an oxidized ferulic acid of the polymeric polysaccharide, and a second site, which provides desired properties to the polymeric polysaccharide matrix, characterized by being obtainable by the method of claim 2.

23. Use of a modified polymeric polysaccharide matrix according to claim 19 or 20 in thickening agents, hydrogels, films, edible coatings or coatings of packaging materials.

24. Use of a product according to claim 21 or 22 for manufacturing packages of food products, animal feed, cosmetics or electronics.

25. A method, modified polymeric polysaccharide matrix, product being coated with a modified polymeric polysaccharide matrix or use according to claim 1, characterized in that the polymeric polysaccharide is a pectin or xylan.
